(19) United States
(12) Patent Application Publication
(10) Pub. No.: US 2024/0334036 A1
(43) Pub. Date: Oct. 3, 2024

(54) IMAGE PICKUP UNIT AND ENDOSCOPE

(71) Applicant: OLYMPUS MEDICAL SYSTEMS CORP., Tokyo (JP)

(72) Inventors: Shuichi TAKIE, Tokyo (JP); Takanori SEKIDO, Sagamihara-shi (JP)

(73) Assignee: OLYMPUS MEDICAL SYSTEMS CORP., Tokyo (JP)

(21) Appl. No.: 18/739,970

(22) Filed: Jun. 11, 2024

Related U.S. Application Data
(63) Continuation of application No. PCT/JP2022/021606, filed on May 26, 2022.

(51) Int. Cl. H04N 23/50; H04N 23/54; H04N 25/79

(52) U.S. Cl. CPC H04N 23/555; H04N 23/54; H04N 25/79

(57) ABSTRACT

A three-dimensional circuit board connected to a back surface of a plane circuit board includes a front surface corresponding to the back surface of the plane circuit board, an upper surface and a lower surface intersecting the front surface, a first connection electrode provided on the front surface, a second connection electrode extended from the first connection electrode to the upper surface and the lower surface, and a resist layer covering the second connection electrode in each of regions on the upper surface and the lower surface and near the front surface.

### CROSS REFERENCE TO RELATED APPLICATION

This application is a continuation application of PCT/JP2022/021606 filed on May 26, 2022, the entire contents of which are incorporated herein by this reference.

### BACKGROUND OF THE INVENTION

### 1. Field of the Invention

The present disclosure relates to an image pickup unit and an endoscope, the image pickup unit being provided inside a distal end portion of an insertion portion of the endoscope and configured to pick up an image of inside of a subject.

### 2. Description of the Related Art

Conventionally, an endoscope for performing various examinations has been widely used in medical and industrial fields. For example, the endoscope can acquire an image of inside of a subject by inserting an insertion portion into the subject.

Typically, an image pickup unit is provided at a distal end of the insertion portion of the endoscope. The image pickup unit includes an image pickup device, and a circuit board connected to the image pickup device. A plurality of signal cables are soldered to the circuit board of the image pickup unit.

Recently, an image pickup unit including a three-dimensional circuit board has been proposed to, for example, simplify work of connecting a signal cable or the like to a circuit board, improve reliability of a connection part of a signal cable or the like, and ensure mounting area of a plurality of components.

For example, International Publication No. 2016/092986 (refer to FIGS. 15 and 16, for example) discloses an image pickup unit including a semiconductor package, a plane circuit board, and an odd-shaped circuit board (three-dimensional circuit board). In this technology of International Publication No. 2016/092986, the odd-shaped circuit board has a first surface (front surface) connected to the plane circuit board. A recessed part for avoiding interference with an electronic component mounted on the plane circuit board is formed at the first surface of the odd-shaped circuit board. In addition, a plurality of connection electrodes constituting a three-dimensional wire are formed at the first surface on both sides of the recessed part. The connection electrodes formed at the first surface are extended to each of a second surface and a third surface adjacent to the first surface. In addition, the connection electrodes formed at the first surface are connected to the plane circuit board through solder. Cables bifurcated from a composite cable are connected to the respective connection electrodes formed at the second and third surfaces through solder.

### SUMMARY OF THE INVENTION

An image pickup unit according to an aspect of the present disclosure includes an image pickup device, a first circuit board connected to a back surface of the image pickup device, and a second circuit board connected to a back surface of the first circuit board, the second circuit board includes a first surface that is a connection surface to the back surface of the first circuit board, a second surface intersecting the first surface, a chamfered part formed between the first surface and the second surface, a first connection electrode provided on the first surface and the chamfered part, a second connection electrode extended from the first connection electrode to the second surface, and a resist layer covering the second connection electrode in a region on the second surface and near the first surface, an end part of the resist layer on the first surface side is set at a boundary between the chamfered part and the second surface, and the first circuit board and the second circuit board are connected to each other by solder provided between the back surface of the first circuit board and the first connection electrode.

An endoscope according to an aspect of the present disclosure includes an insertion portion that is inserted into a subject, and an image pickup unit provided at a distal end portion of the insertion portion, the image pickup unit includes an image pickup device, a first circuit board connected to a back surface of the image pickup device, and a second circuit board connected to a back surface of the first circuit board, the second circuit board includes a first surface that is a connection surface to the back surface of the first circuit board, a second surface intersecting the first surface, a chamfered part formed between the first surface and the second surface, a first connection electrode provided on the first surface and the chamfered part, a second connection electrode extended from the first connection electrode to the second surface, and a resist layer covering the second connection electrode in a region on the second surface and near the first surface, an end part of the resist layer on the first surface side is set at a boundary between the chamfered part and the second surface, and the first circuit board and the second circuit board are connected to each other by solder provided between the back surface of the first circuit board and the first connection electrode.

### BRIEF DESCRIPTION OF THE DRAWINGS

FIG. 1 relates to an embodiment of the present disclosure and is an exterior diagram illustrating an endoscope system;

FIG. 2 relates to the embodiment of the present disclosure and is a perspective view of an image pickup unit when viewed from obliquely backward right;

FIG. 3 relates to the embodiment of the present disclosure and is an exploded perspective view illustrating a main part of the image pickup unit;

FIG. 4 relates to the embodiment of the present disclosure and is a plan view of the image pickup unit when viewed from a back surface side;

FIG. 5 relates to the embodiment of the present disclosure and is a V-V cross-sectional view of FIG. 4;

FIG. 6 relates to the embodiment of the present disclosure and is a main-part cross-sectional view of the image pickup unit at thermal expansion;

FIG. 7 relates to the embodiment of the present disclosure and is an enlarged cross-sectional view of a connection part of a plane circuit board and a three-dimensional circuit board;

FIG. 8 relates to a first modification and is a main-part cross-sectional view of the image pickup unit;

FIG. 9 relates to a second modification and is a plan view of the image pickup unit when viewed from an upper surface side;

FIG. 10 relates to the second modification and is an X-X cross-sectional view of FIG. 9;

FIG. 11 relates to a third modification and is a perspective view of the image pickup unit when viewed from obliquely backward right; and

FIG. 12 relates to the third modification and is a plan view of the image pickup unit when viewed from an upper surface side.

### DETAILED DESCRIPTION OF THE PREFERRED EMBODIMENT

The present disclosure will be described below with an illustrated embodiment.

Each drawing used in the following description is schematically illustrated, and dimensional relations, scaling, and the like among members are illustrated differently for each constituent component in some cases to indicate the constituent component in a size enough to allow recognition on the drawing. Thus, in the present disclosure, for example, the number of constituent components illustrated in each drawing, a shape of each constituent component, a size ratio of the constituent components, and a relative positional relation among the constituent components are not limited to illustrated forms.

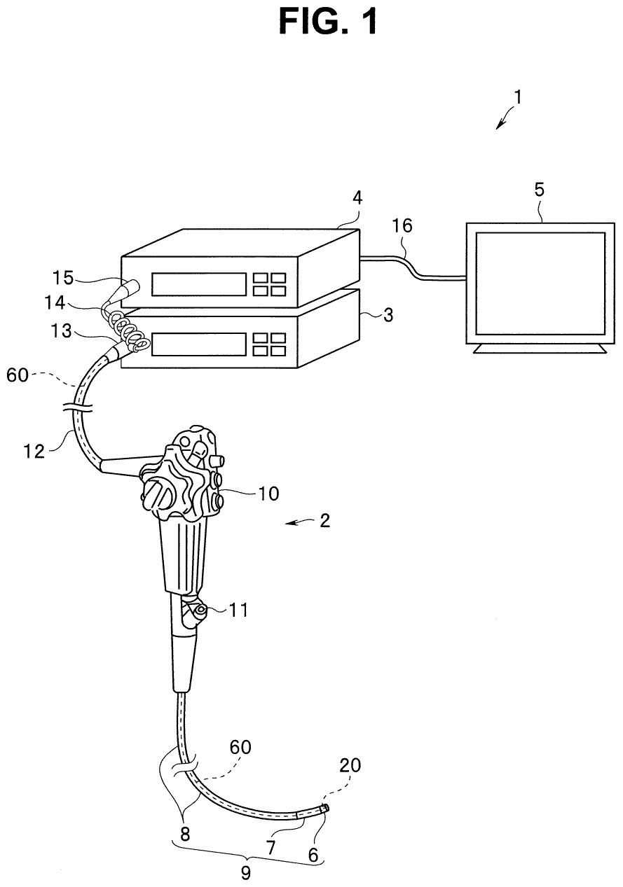

Before a detailed configuration of an image pickup unit of an embodiment of the present disclosure is described, an entire schematic configuration of an endoscope system including an endoscope to which the image pickup unit of the present embodiment is applied will be briefly described below with reference to FIG. 1.

FIG. 1 is an exterior diagram illustrating an endoscope system including an endoscope to which the image pickup unit of the embodiment of the present disclosure is applied. The endoscope system has substantially the same basic configuration as a conventional endoscope system. Thus, the following description only includes schematic description of each constituent member in the endoscope system.

As illustrated in FIG. 1, an endoscope system 1 including an endoscope to which the image pickup unit of the present embodiment is applied includes an endoscope 2, a light source device 3, a video processor 4, and a display device 5.

As illustrated in FIG. 1, the endoscope 2 includes an insertion portion 9 having a substantially elongated pipe shape, an operation portion 10 connected to a proximal end portion of the insertion portion 9, and a universal code 12 extended from the operation portion 10.

The insertion portion 9 of the endoscope 2 is constituted by a distal end portion 6, a bending portion 7, and a flexible tube portion 8, which are continuously provided in order from a distal end side. The proximal end portion of the insertion portion 9 is connected to the operation portion 10.

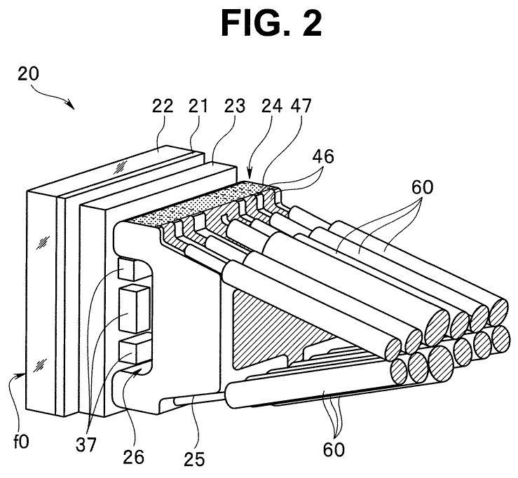

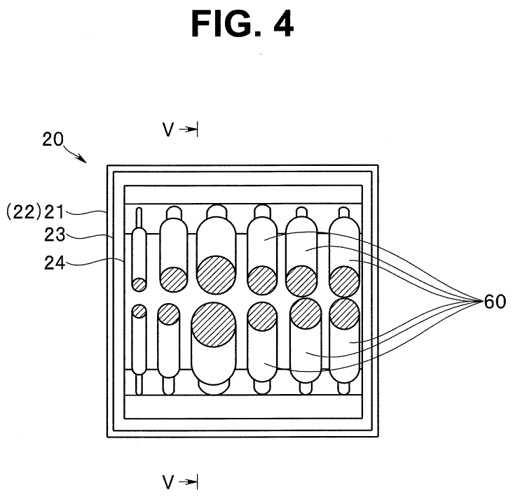

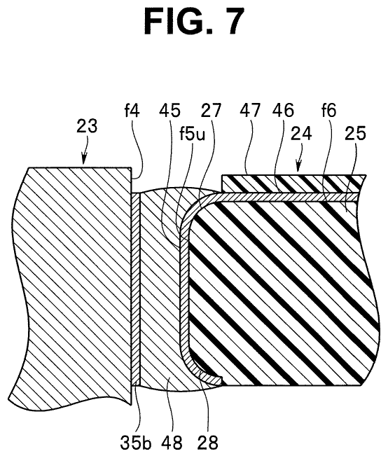

An image pickup unit 20 of the present embodiment is disposed inside the distal end portion 6. A detailed configuration of the image pickup unit 20 of the present embodiment will be described later (refer to FIGS. 2 to 7).

The operation portion 10 includes a forceps port 11 having an opening for inserting a treatment instrument or the like, an operation portion body constituting a grasping portion, and a plurality of operation members that are provided on an outer surface of the operation portion body and with which various operations of the endoscope 2 are performed.

The forceps port 11 provided at the operation portion 10 constitutes a proximal-end-side opening part of a treatment instrument channel (not illustrated) inserted and disposed between the operation portion 10 and a distal-end-side opening part of the distal end portion 6 of the insertion portion 9.

The universal code 12 is a tubal member extending from a side of the operation portion 10. A scope connector 13 is provided at an extended end site of the universal code 12. The scope connector 13 is connected to the light source device 3.

The light source device 3 is a device that supplies illumination light to an illumination unit (not illustrated) provided inside the distal end portion 6 of the insertion portion 9 of the endoscope 2. Illumination light emitted from the light source device 3 is transferred from the scope connector 13 to the distal end portion 6 through an optical fiber cable (not illustrated) inserted and disposed through the universal code 12, the operation portion 10, and the insertion portion 9. Then, the illumination light transmits through an illumination optical member provided at a front surface of the distal end portion 6 and is emitted toward an observation target object on a front side of the distal end portion 6.

A scope cable 14 extends toward a side from the scope connector 13. An electric connector portion 15 is provided at a distal end site of the scope cable 14. The electric connector portion 15 is connected to the video processor 4.

The video processor 4 is a control device that controls the entire endoscope system 1. In this case, the video processor 4 includes, for example, a signal processing circuit configured to receive an image pickup signal from the image pickup unit 20 provided inside the distal end portion 6 of the insertion portion 9 of the endoscope 2 and perform predetermined signal processing, a control processing circuit configured to output a control signal that drives the image pickup unit 20, or the like.

A signal transmission cable (hereinafter simply referred to as a cable) 60 electrically connects the video processor 4 and the image pickup unit 20. Thus, the cable 60 is inserted and disposed through the electric connector portion 15, the universal code 12, the operation portion 10, and the distal end portion 6 of the insertion portion 9. With this configuration, an image pickup signal outputted from the image pickup unit 20, a control signal outputted from the video processor 4, and the like are transferred between the image pickup unit 20 and the video processor 4 through the cable 60. Note that a form of the cable 60 is, for example, a composite cable formed by bundling a plurality of cables and covered with an external skin shield, an external skin tube, or the like.

A video cable 16 connects the video processor 4 and the display device 5. The video cable 16 transmits an image signal, a control signal, and the like outputted from the video processor 4 to the display device 5.

The display device 5 receives image and control signals outputted from the video processor 4 and displays an endoscope image and various kinds of information in a predetermined form corresponding to a display form in accordance with the received control signal. The endoscope system 1 including the endoscope 2 to which the image pickup unit 20 of the present embodiment is applied is schematically configured as described above. Note that the other configuration of the endoscope system 1 is substantially the same as that of a conventional endoscope system of the same kind.

The configuration of the image pickup unit of the present embodiment will be described below in detail with reference to FIGS. 2 to 7. FIG. 2 is a perspective view illustrating appearance of the image pickup unit of the present embodiment. Reference sign f0 illustrated in FIG. 2 denotes a front surface region of the image pickup unit. Unless otherwise stated, up, down, right, and left directions in the following description are up, down, right, and left directions when viewed from a position facing the front surface region f0 of the image pickup unit 20. Moreover, the up, down, right, and left directions of the image pickup unit 20 correspond to the up, down, right, and left directions of an endoscope image displayed on the display device 5.

The image pickup unit 20 includes an image pickup device 21, a cover glass 22, a plane circuit board 23 as a first circuit board, and a three-dimensional circuit board 24 as a second circuit board.

The image pickup device 21 receives an optical image of the observation target object, which is formed by an image forming optical unit (not illustrated). In addition, the image pickup device 21 performs predetermined photoelectric conversion processing on the received optical image. Accordingly, the image pickup device 21 generates an image signal of the observation target object.

The image pickup device 21 is an image sensor of a typical form, such as a charge coupled device (CCD) or a complementary metal oxide semiconductor (CMOS).

The cover glass 22 that is a protection glass is provided on a front surface (in other words, light-receiving surface) f1 side of the image pickup device 21. An image forming optical lens unit (not illustrated) is disposed on the front side (distal end side) of the image pickup device 21 in a state in which the image pickup unit 20 is disposed inside the distal end portion 6 of the endoscope 2. In this case, the image forming optical lens unit is disposed such that its optical axis (not illustrated) is aligns with a substantially central axis of the image pickup device 21. The light-receiving surface f1 of the image pickup device 21 is disposed in parallel to a surface orthogonal to the optical axis of the image forming optical lens unit. With these configurations, light from the observation target object, which is condensed through the image forming optical lens unit, is incident on the front surface f0 of the cover glass 22, transmits through the cover glass 22, and forms an image on the light-receiving surface f1 of the image pickup device 21.

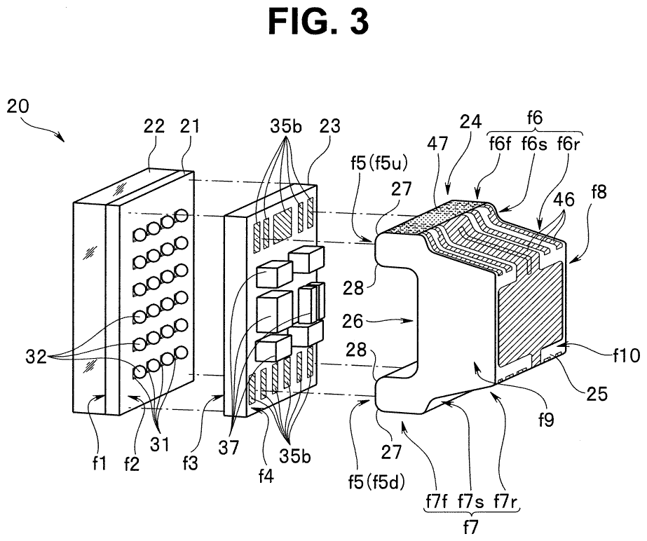

As illustrated in FIG. 3, a plurality of connection electrodes 31 are provided on a back surface f2 (surface on a side opposite the front surface f1) of the image pickup device 21. In addition, solder 32 formed in a bump shape is provided at each connection electrode 31.

The plane circuit board 23 is a circuit board having a flat plate shape as a whole. The plane circuit board 23 is, for example, a multi-layer structure substrate formed by stacking a plurality of substrates. The substrates stacked in the plane circuit board 23 are, for example, ceramics substrates, epoxy glass substrates, flexible substrates, glass substrates, and silicone substrates. Note that the plane circuit board 23 has the same structure as that conventionally typically used. Thus, detailed description of the plane circuit board 23 is omitted. The plane circuit board 23 is provided substantially in parallel to the image pickup device 21.

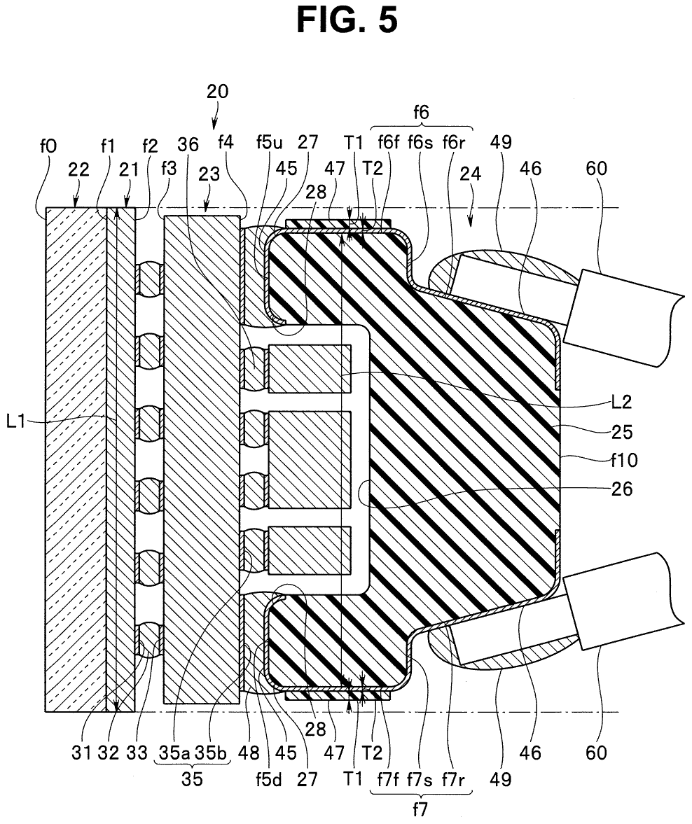

As illustrated in FIG. 5, a plurality of connection electrodes 33 corresponding to the respective connection electrodes 31 of the image pickup device 21 are provided on a front surface f3 of the plane circuit board 23. Each connection electrode 33 is connected to the corresponding connection electrode 31 as the solder 32 fuses. Accordingly, the plane circuit board 23 is electrically and mechanically connected to the image pickup device 21.

As illustrated in FIGS. 3 and 5, a plurality of connection electrodes 35 are provided on a back surface f4 (surface on a side opposite the front surface f3) of the plane circuit board 23.

Among the connection electrodes 35, connection electrodes 35a disposed in a central region of the back surface f4 of the plane circuit board 23 in the up-down direction are connection electrodes for mounting a plurality of electronic components 37 on the plane circuit board 23. Each connection electrode 35a is connected through solder 36 to, for example, a passive component such as a capacitor, a resistor, or a coil, or an active component such as a transistor, a diode, or a drive IC.

Among the connection electrodes 35, connection electrodes 35b disposed in an edge part region of the back surface f4 of the plane circuit board 23 in the up-down direction are connection electrodes for connection to the three-dimensional circuit board 24.

The three-dimensional circuit board 24 is what is called an odd-shaped circuit board having a three-dimensional structure. The three-dimensional circuit board 24 is, for example, a molded interconnect device (MID) substrate obtained by forming a three-dimensional wire on an injection-molded resin component (substrate body 25). Note that the three-dimensional circuit board 24 may be other than the MID substrate and may be, for example, a ceramics substrate, an epoxy glass substrate, a glass substrate, or a silicone substrate.

The substrate body 25 of the three-dimensional circuit board 24 has a front surface f5 as a first surface. The front surface f5 is set as a connection surface for connecting the three-dimensional circuit board 24 to the back surface f4 of the plane circuit board 23.

In the present embodiment, the front surface f5 of the substrate body 25 is divided into a front surface f5u on the upper side and a front surface f5d on the lower side by a recessed part 26. The recessed part 26 is a recessed part for avoiding interference between the electronic components 37 mounted on the plane circuit board 23 and the three-dimensional circuit board 24. The recessed part 26 is formed at a front part of the substrate body 25 and at a substantially central part thereof in the up-down direction.

The substrate body 25 has a pair of an upper surface f6 and a lower surface f7 formed as second surfaces on the upper and lower sides. The upper surface f6 and the lower surface f7 are formed along a pair of upper and lower sides, respectively, parallel to each other at edge parts of the front surface f5. Specifically, the upper surface f6 is formed along a top side of the front surface f5u. The lower surface f7 is formed along a bottom side of the front surface f5d. The upper surface f6 and the lower surface f7 are each extended in a direction intersecting the front surface f5 (for example, a direction orthogonal thereto).

The upper surface f6 has a front part region f6f and a back part region for. The front part region f6f is constituted by a surface substantially orthogonal to the front surface f5. The back part region for is constituted by a surface tilted toward inside of the substrate body 25 from the front side (front surface side) of the substrate body 25 to the back side (back surface side) thereof. The front part region f6f and the back part region for are continuous with each other with a step surface f6s interposed therebetween.

Similarly, the lower surface f7 has a front part region f7f and a back part region f7r. The front part region f7f is constituted by a surface substantially orthogonal to the front surface f5. The back part region f7r is constituted by a surface tilted toward inside of the substrate body 25 from the front side (front surface side) of the substrate body 25 to the back side (back surface side) thereof. The front part region f7f and the back part region f7r are continuous with each other with a step surface f7s interposed therebetween.

The substrate body 25 also has a pair of a left side surface f8 and a right side surface f9 formed as third surfaces on the right and left sides. The left side surface f8 and the right side surface f9 are each constituted by a surface intersecting the front surface f5, the upper surface f6, and the lower surface f7. More specifically, for example, the left side surface f8 and the right side surface f9 are each constituted by a surface orthogonal to the front surface f5, the front part region f6f of the upper surface f6, and the front part region f7f of the lower surface f7.

The substrate body 25 also includes a back surface f10. The back surface f10 is constituted by, for example, a surface parallel to the front surface f5. The back surface f10 is also constituted by a surface intersecting the upper surface f6, the lower surface f7, and the right and left side surfaces f8 and f9.

For example, as illustrated in FIGS. 5 to 7, a chamfered part 27 is provided at each of a boundary between the front surface f5u and the upper surface f6 and a boundary between the front surface f5d and the lower surface f7 in the substrate body 25 of the present embodiment. In addition, a chamfered part 28 is provided at each of a boundary between the front surface f5u and the recessed part 26 and a boundary between the front surface f5d and the recessed part 26.

A plurality of first connection electrodes 45 are formed on the front surface f5 (front surfaces f5u and f5d) of the substrate body 25. The first connection electrodes 45 on the front surfaces f5u and f5d are disposed at positions corresponding to the respective connection electrodes 35b on the plane circuit board 23. Each first connection electrode 45 is extended from the front surface f5u or f5d to the chamfered part 27 or 28.

In addition, a plurality of second connection electrodes 46 are formed on each of the upper surface f6 and the lower surface f7 of the substrate body 25. In the present embodiment, each second connection electrode 46 is formed by, for example, extending a three-dimensional wire forming a first connection electrode 45 on the front surface f5u or f5d to each of the upper surface f6 and the lower surface f7. In other words, the second connection electrodes 46 are constituted by a set of three-dimensional wires electrically connected to the first connection electrodes 45, respectively.

Part of each second connection electrode 46 formed on the upper surface f6 and part of each second connection electrode 46 formed on the lower surface f7 are extended to the back surface f10 of the substrate body 25. The second connection electrodes 46 extended from the upper surface f6 and the second connection electrodes 46 extended from the lower surface f7 are electrically connected to each other on the back surface f10.

Note that three-dimensional wires forming the first connection electrodes 45 and the second connection electrodes 46 as described above may preferably have, for example, a thickness of 20 μm approximately.

In addition, a resist layer (solder resist layer) 47 covering part of the second connection electrodes 46 is formed on each of the upper surface f6 and the lower surface f7 of the substrate body 25. Each resist layer 47 is formed in a region on the upper surface f6 or the lower surface f7 and near the front surface f5u or f5d.

More specifically, each resist layer 47 is constituted by, for example, a strip-shaped resin layer extending in a direction intersecting the second connection electrodes 46 on the front part region f6f of the upper surface f6 or the front part region f7f of the lower surface f7. In this case, a front edge of each resist layer 47 formed on the upper surface f6 or the lower surface f7 may preferably be set at a position matching an end part of the chamfered part 27 between the front surface f5u and the upper surface f6 or an end part of the chamfered part 27 between the front surface f5d and the lower surface f7.

Each resist layer 47 may preferably have a thickness of 30 μm approximately, for example. Such a resist layer 47 can be excellently formed by using, for example, a dispenser. The thickness of the resist layer 47 can be controlled by adjusting application time and discharge pressure of the dispenser.

In the three-dimensional circuit board 24 thus configured, each first connection electrode 45 is connected to the corresponding connection electrode 35b on the plane circuit board 23 through solder 48.

In addition, a plurality of cables 60 are connected to the second connection electrodes 46, respectively, through solder 49.

The solder 48 in paste form is applied to, for example, each connection electrode 35b of the plane circuit board 23 by a dispenser or the like at connection between the connection electrode 35b and the corresponding first connection electrode 45. In addition, each first connection electrode 45 of the three-dimensional circuit board 24 is positioned relative to the corresponding connection electrode 35b to which the solder 48 is applied. Then, thermal treatment is performed on the solder 48. Accordingly, each first connection electrode 45 is electrically and mechanically connected to the corresponding connection electrode 35b through the solder 48. In this manner, the three-dimensional circuit board 24 is connected to the plane circuit board 23 through the solder 48.

In this case, the solder 48 melted by the thermal treatment is stemmed by the resist layers 47. Thus, the solder 48 is accurately prevented from flowing to the second connection electrode 46 side. Accordingly, a sufficient amount of the solder 48 is held between each connection electrode 35b and the corresponding first connection electrode 45 so that the first connection electrode 45 is solidly connected to the connection electrode 35b.

In the image pickup unit 20 configured as described above, a dimension of each component in a direction orthogonal to the pair of resist layers 47 is defined as described below.

Specifically, as illustrated in FIG. 5, at room temperature (for example, 10° C. to 30° C.), the dimension of the image pickup device 21 (and the cover glass 22) is defined as L1, the dimension of the substrate body 25 is defined as L2, the thickness of each resist layer 47 is defined as T1, and the thickness of each second connection electrode 46 is defined as T2.

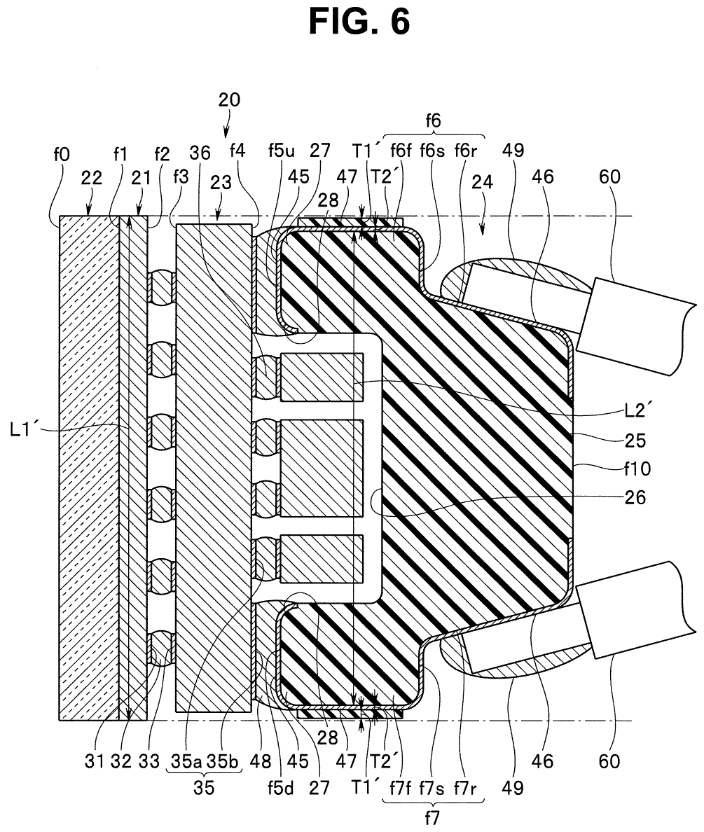

In addition, as illustrated in FIG. 6, at high temperature (for example, 60° C. to 150° C.), the dimension of the image pickup device 21 (and the cover glass 22) is defined as L1′, the dimension of the substrate body 25 is defined as L2′, the thickness of each resist layer 47 is defined as T1′, and the thickness of each second connection electrode 46 is defined as T2′. In the present embodiment, the above-described high temperature corresponds to cure temperature of a bonding agent made of thermosetting resin or the like.

When the dimensions are defined in this manner, relations among the dimensions may preferably be designed to satisfy relations of Expressions (1) and (2) below.

$$L 1 > L 2 + 2 ( T 1 + T 2 ) \qquad ( 1 )$$

$$L {1}^{′} > L {2}^{′} + 2 ( T {1}^{′} + T {2}^{′} ) \qquad ( 2 )$$

Specifically, the three-dimensional circuit board 24 may preferably have dimensions with which the three-dimensional circuit board 24 is constantly positioned inside a projection plane of the image pickup device 21 when the three-dimensional circuit board 24 is projected in a direction of an image pickup optical axis.

This designing of dimensions improves, for example, assembly easiness when the image pickup unit 20 is assembled to a distal end frame of the distal end portion 6 of the endoscope 2 or the like. Specifically, for example, when the image pickup unit 20 is sealed to the distal end frame or the like by using thermosetting resin or other material, damage or other defect due to interference of the three-dimensional circuit board 24 being thermally expanded with the distal end frame or the like is prevented, which improves, for example, yield at assembly.

Note that the material of the substrate body 25 of the three-dimensional circuit board 24 may preferably be resin having a thermal expansion coefficient of 10 to 60×10⁻⁶/K approximately.

In this case, for example, when the substrate (substrate body) has a dimension of 3 mm or smaller in one direction, the dimension along with change in temperature of the substrate from room temperature to 150° C. is as described below, for example.
Example 1 Dimension at room temperature: 1.000 [mm]→Dimension at heating: 1.004 [mm] approximately
Example 2 Dimension at room temperature: 3.000 [mm]→Dimension at heating: 3.012 [mm] approximately

Based on them, the three-dimensional circuit board 24 can be designed by using, for example, a relation of Expression (3) below as a guide.

$$L 2 + 2 ( T 1 + T 2 ) + ( dimension expansion ) \geq L {2}^{′} + 2 ( T {1}^{′} + T {2}^{′} ) \qquad ( 3 )$$

According to such an embodiment, the three-dimensional circuit board 24 connected to the back surface of the plane circuit board 23 includes the front surface f5 corresponding to the back surface of the plane circuit board 23, the upper surface f6 and the lower surface f7 intersecting the front surface f5, the first connection electrodes 45 provided on the front surface f5, the second connection electrodes 46 extended from the first connection electrodes 45 to the upper surface f6 and the lower surface f7, and the resist layers 47 covering the second connection electrodes 46 in the regions on the upper surface f6 and the lower surface f7 and near the front surface f5. The plane circuit board 23 and the three-dimensional circuit board 24 are connected to each other by the solder 48 provided between each connection electrode 35b of the plane circuit board 23 and the corresponding first connection electrode 45 of the three-dimensional circuit board 24.

Accordingly, outflow of the solder 48 can be prevented and the three-dimensional circuit board 24 can be accurately bonded to the plane circuit board 23.

Specifically, the three-dimensional circuit board 24 includes the resist layers 47 covering the second connection electrodes 46 in the regions on the upper surface f6 and the lower surface f7 and near the front surface f5. With this configuration, the solder 48 being melted is prevented from flowing from the first connection electrode 45 side to the second connection electrode 46 side when the three-dimensional circuit board 24 is connected to the plane circuit board 23 by using the solder 48.

Accordingly, a sufficient amount of the solder 48 can be held between each connection electrode 35b and the corresponding first connection electrode 45 so that the first connection electrode 45 is solidly connected to the connection electrode 35b.

In this case, the three-dimensional circuit board 24 includes the chamfered parts 27 between the front surface f5u and the upper surface f6 and between the front surface f5d and the lower surface f7. The front edge of each resist layer 47 is formed at the end part of the corresponding chamfered part 27. With this configuration, bonding area of the solder 48 to the first connection electrodes 45 is increased by an amount of increase in surface area due to each chamfered part 27 made of a curved surface. Accordingly, strength of connection of the three-dimensional circuit board 24 to the plane circuit board 23 is improved.

Moreover, breaking between the first connection electrodes 45 and the second connection electrodes 46 can be prevented since the chamfered part 27 is formed at the boundary between the front surface f5u and the upper surface f6.

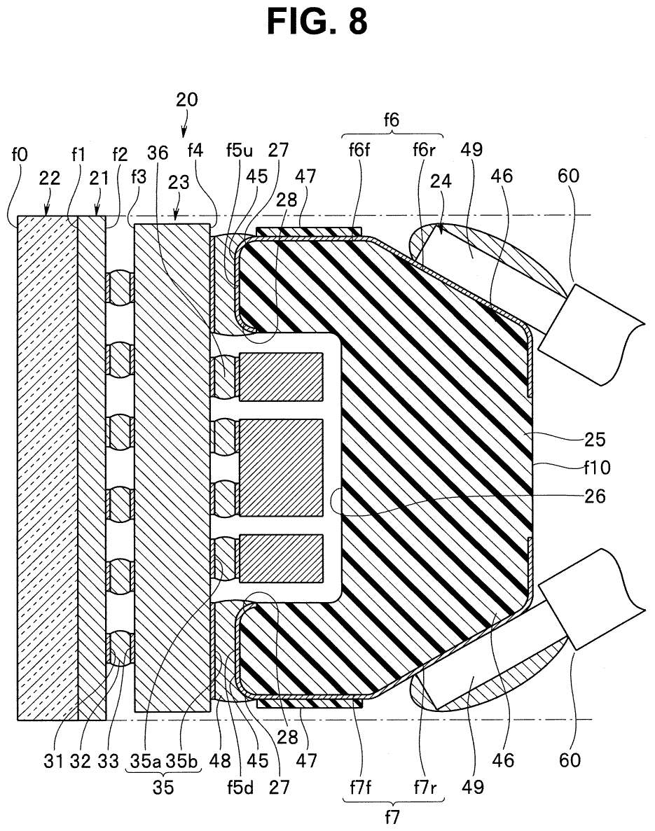

A first modification of the present embodiment will be described below with reference to FIG. 8.

In the present modification, the front part region f6f and the back part region for on the upper surface f6 of the three-dimensional circuit board 24 are continuous with each other without a step surface interposed therebetween.

Similarly, the front part region f7f and the back part region f7r on the lower surface f7 of the three-dimensional circuit board 24 are continuous with each other without a step surface interposed therebetween.

In such a configuration, slopes of the back part regions for and f7r are set to be larger than those of the upper surface f6 and the lower surface f7 having step surfaces. With this setting, the three-dimensional circuit board 24 including the cable 60 is positioned inside the projection plane of the image pickup device 21 when the cable 60 is connected to each second connection electrode 46.

With such a configuration of the first modification as well, it is possible to achieve substantially the same effects as those of the above-described embodiment.

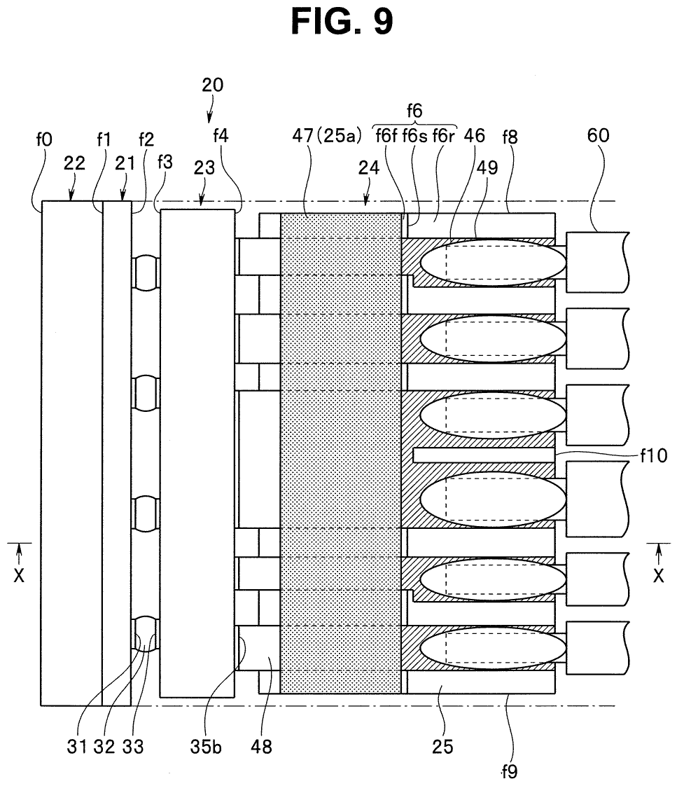

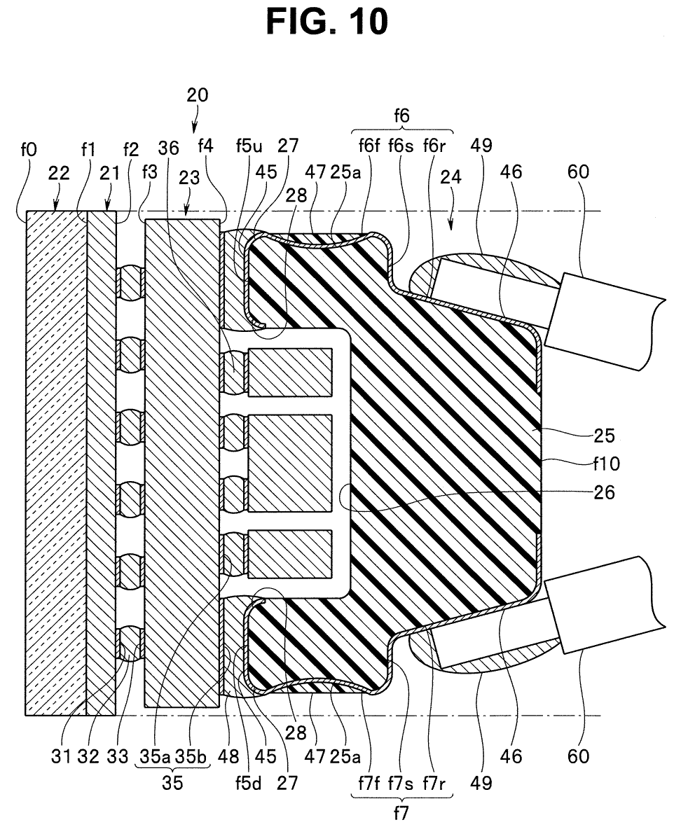

A second modification of the present embodiment will be described below with reference to FIGS. 9 to 11.

In the present modification, a groove part 25a is provided in a direction intersecting the second connection electrodes 46 in the front part region f6f of the upper surface f6 of the three-dimensional circuit board 24.

Similarly, another groove part 25a is provided in a direction intersecting the second connection electrodes 46 in the front part region f7f of the lower surface f7 of the three-dimensional circuit board 24.

The resist layers 47 are formed in the respective groove parts 25a.

In such a second modification, when resin solution or the like is applied on the upper surface f6 and the lower surface f7 to form the resist layers 47, the resin solution or the like stays in the groove parts 25a due to surface tension of the resin solution or the like, and thus it is easy to control ranges in which the resist layers 47 are formed.

In this case, each groove part 25a may have, for example, a section in a semi-elliptical shape with a depth of 50 μm approximately. The thickness of each resist layer 47 formed in the groove part 25a may preferably be smaller than the depth of the groove part 25a.

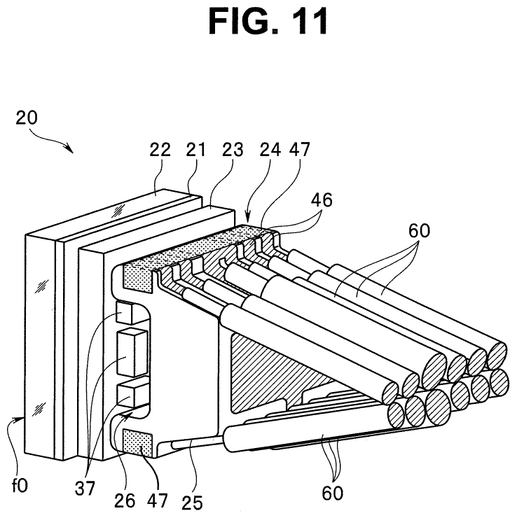

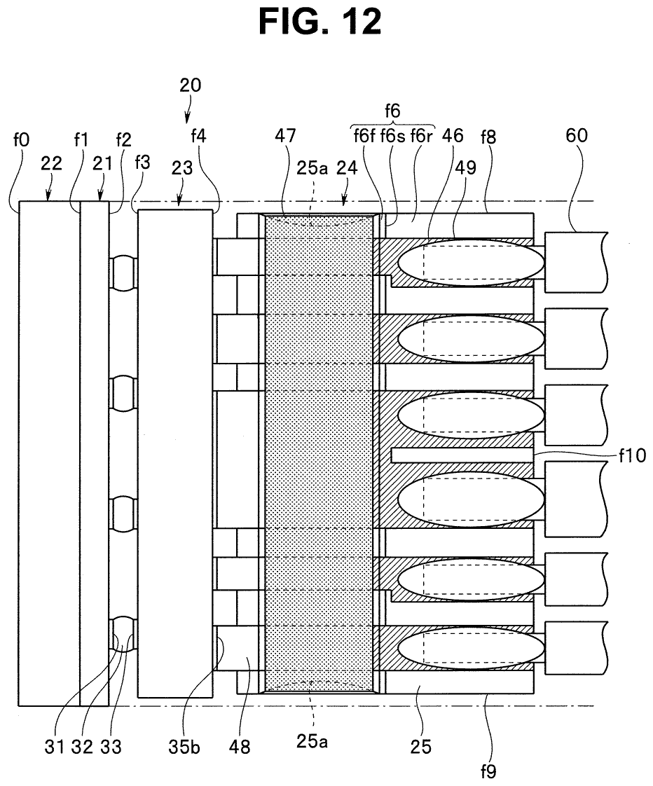

A third modification of the present embodiment will be described below with reference to FIGS. 11 and 12.

The present modification relates to a configuration in which the groove parts 25a and the resist layers 47 described in the above-described second modification are extended to the right and left side surfaces f8 and f9.

With this configuration, it is possible to more effectively prevent outflow of resin solution when the resist layers 47 are formed. Moreover, it is possible to prevent overflowing of the resist layers 47 beyond dimensions of the image pickup device 21.

Note that the present disclosure is not limited to each above-described embodiment but may be provided with various kinds of deformation and change, which are included in the technical scope of the present disclosure. Moreover, it is of course possible to combine configurations of the above-described embodiment and modifications as appropriate.

What is claimed is:

1. An image pickup unit comprising:
an image pickup device;
a first circuit board connected to a back surface of the image pickup device; and
a second circuit board connected to a back surface of the first circuit board, wherein
the second circuit board includes a first surface that is a connection surface to the back surface of the first circuit board, a second surface intersecting the first surface, a chamfered part formed between the first surface and the second surface, a first connection electrode provided on the first surface and the chamfered part, a second connection electrode extended from the first connection electrode to the second surface, and a resist layer covering the second connection electrode in a region on the second surface and near the first surface,
an end part of the resist layer on the first surface side is set at a boundary between the chamfered part and the second surface, and
the first circuit board and the second circuit board are connected to each other by solder provided between the back surface of the first circuit board and the first connection electrode.

2. The image pickup unit according to claim 1, wherein
a pair of the second surfaces are formed along a pair of sides parallel to each other at edge parts of the first surface,
the second connection electrode and the resist layer are provided on each of the pair of second surfaces,
a relation of L1>L2+2 (T1+T2) and a relation of L1′>L2′+2 (T1′+T2′) are satisfied where
L1 represents a dimension of the image pickup device in a direction orthogonal to the pair of sides at room temperature and L1′ represents a dimension of the image pickup device at heating to 60° C. to 150° C.,
L2 represents a dimension between the pair of second surfaces of the second circuit board at room temperature and L2′ represents a dimension between the pair of the second surfaces at heating to 60° C. to 150° C.,
T1 represents a dimension of the resist layer at room temperature and T1′ represents a dimension of the resist layer at heating to 60° C. to 150° C., and
T2 represents a dimension of the second connection electrode at room temperature and T2′ represents a dimension of the second connection electrode at heating to 60° C. to 150° C.

3. The image pickup unit according to claim 1, wherein
the second surface includes a groove part, and
the resist layer is provided at the groove part.

4. The image pickup unit according to claim 1, wherein the resist layer is extended to a third surface intersecting the first surface and the second surface.

5. The image pickup unit according to claim 3, wherein
the groove part is extended to a third surface intersecting the first surface and the second surface, and
a thickness of the resist layer formed at the groove part is smaller than a depth of the groove part.

6. An endoscope comprising:
an insertion portion configured to be inserted into a subject; and
an image pickup unit provided at a distal end portion of the insertion portion, wherein
the image pickup unit includes an image pickup device, a first circuit board connected to a back surface of the image pickup device, and a second circuit board connected to a back surface of the first circuit board,
the second circuit board includes a first surface that is a connection surface to the back surface of the first circuit board, a second surface intersecting the first surface, a chamfered part formed between the first surface and the second surface, a first connection electrode provided on the first surface and the chamfered part, a second connection electrode extended from the first connection electrode to the second surface, and a resist layer covering the second connection electrode in a region on the second surface and near the first surface,
an end part of the resist layer on the first surface side is set at a boundary between the chamfered part and the second surface, and
the first circuit board and the second circuit board are connected to each other by solder provided between the back surface of the first circuit board and the first connection electrode.
